(19) United States
(12) Patent Application Publication
(10) Pub. No.: US 2018/0303413 A1
(43) Pub. Date: Oct. 25, 2018

(54) SYSTEM AND METHOD FOR MONITORING AND DETERMINING A MEDICAL CONDITION OF A USER

(71) Applicant: Healthymize Ltd, Julis (IL)

(72) Inventors: Shadi HASSAN, Julis (IL); Daniel ARONOVICH, Kibbutz Hannaton (IL)

(73) Assignee: Healthymize Ltd, Julis (IL)

(21) Appl. No.: 15/769,071

(22) PCT Filed: Oct. 19, 2016
(86) PCT No.: PCT/IL2016/051131

§ 371 Date: Apr. 18, 2018

Related U.S. Application Data
(60) Provisional application No. 62/243,680, filed on Oct. 20, 2015.
(60) Provisional application No. 62/252,587, filed on Nov. 9, 2015.
(60) Provisional application No. 62/274,598, filed on Jan. 4, 2016.

(51) Int. Cl. A61B 5/00; G10L 25/66; A61B 5/08; A61B 7/00

(52) U.S. Cl. CPC A61B 5/4833; G10L 25/66; A61B 5/0816; A61B 5/0022; A61B 7/003; A61B 5/4842; A61B 5/6898; A61B 5/0823

(57) ABSTRACT

A system and method for monitoring disease, such as lung disease via a communication device of a user is disclosed. The system and method may include a communication device, such as a smartphone, a laptop, a smartwatch and the like, and may including a memory and a processor configured to: obtain information related to a user's medical condition; determine, while the user is using the communication device, respiratory characteristics of the user based on a recording of a respiration of the user; and determine a progress of a disease of the user based on the user's medical condition and based on comparing the characteristics to a reference set of characteristics. The reference set of characteristics may be created, by the processor, by obtaining signals related to the user's breathing while the user uses the communication device.

### FIELD OF THE INVENTION

The present invention relates generally to home tele-monitoring systems and methods for monitoring and determining a medical condition of a user and/or patient. More specifically, the present invention relates to monitoring and determining a medical condition based on a breathing of a user and/or other respiratory aspects.

### BACKGROUND OF THE INVENTION

Lung Diseases (LD), Obstructive Lung Diseases (OLD), Chronic obstructive pulmonary disease (COPD) and Asthma, as well as other diseases such as heart failure and kidney failure, are known as a major public health problem worldwide.

Known systems and methods include a spectral analysis of a sputum sample collected from the human and determining the state or severity of COPD based on comparing the spectra produced by the analysis to a reference. Other systems and methods known in the art use Fourier transform infrared spectroscopy (FTIR) in order to monitor and predict COPD deterioration of flare-ups.

Some systems and methods known in the art monitor a subject suffering from a chronic medical condition and predict physiological changes which could affect the care of the subject. Examples of such chronic diseases include heart failure, chronic obstructive pulmonary disease, asthma, and diabetes. Monitoring may include measurements of respiratory movements, which can then be analyzed for evidence of changes in respiratory rate, or monitoring for events such as hypopneas, apneas and periodic breathing. Monitoring may be augmented by the measurement of nocturnal heart rate in conjunction with respiratory monitoring. Additional physiological measurements may also be taken, e.g., subjective symptom data, blood pressure, blood oxygen levels, and various molecular markers. Systems and methods for measurements detection of respiratory patterns and heart rate are also known.

There is a long felt unmet need in the art for an effective home monitoring system and/or method that do not require active participation of the patient and/or medical staff and that further enable monitoring and determining a medical condition of a user, in particular, systems and/or methods that enable early pulmonary diseases diagnosis, particularly non-invasive characterization of COPD severity, monitoring of COPD status over time, and early recognition of a COPD ‘flare-up’ for prompt institution of therapy.

### SUMMARY OF THE INVENTION

Embodiments of the present invention provide a system and method for monitoring disease, such as lung disease via a communication device of a user. A system and a method according to some embodiments, may comprise a communication device including a memory and a processor, the processor may be configured to: obtain information related to a user's medical condition; determine, while the user is using the communication device, respiratory characteristics of the user based on a recording of a respiration of the user; and determine a progress of a disease of the user based on the user's medical condition and based on comparing the characteristics to a reference set of characteristics. The reference set of characteristics may be created, by the processor, by obtaining signals related to the user's breathing while the user uses the communication device.

According to some embodiments, the processor may be further configured to determine characteristics of a normal breathing of the user by periodically obtaining respiratory characteristics of the user; and create the reference set of characteristics based on the characteristics of a normal breathing of the user.

According to some embodiments the communication device may be one of: a laptop, a computer, a tablet, a kiosk, a smart phone, a smart watch and a telephone.

According to some embodiments the respiratory characteristics may include characteristics of at least one of: an inhalation, an exhalation, a breathing cycle, a respiratory rate, wheezing, coughs, and lung sounds.

According to some embodiments the information related to the user's medical condition may include at least on of: a physical symptom, physiological data, physical data, a medical history and use of medications.

According to some embodiments the reference set of characteristics may include a breathing frequency curve.

According to some embodiments the processor may be further configured to: associate the progress of the disease with a score; and select to perform, based on the score, at least one action.

According to some embodiments the processor may be further configured to send the respiratory characteristics of the user to a server and the server may be configured to: rank the breathing characteristics according to a ranking scale; and based on the rank, select to send a message to at least one of: a physician, a medical institute, a medical stuff member, a caregiver, a family member of the user, and the user.

According to some embodiments the processor is further configured to generate an alarm using, for example, at least one of: a display of the communication device, a speaker of the communication device, a vibration unit included in the communication device and a network interface unit included in the communication device.

According to some embodiments the processor may be further configured to create the reference set of characteristics by: instructing the user to breath normally; and obtaining respiratory characteristics of the user.

According to some embodiments the processor may be further configured to alert the user based on the determined progress of the disease.

According to some embodiments the processor may be further configured to: generate a baseline based on the respiratory characteristics and based on at least one of: symptoms exhibited by the user and vital signs of the user; and determine a progress of a disease based on comparing symptoms exhibited by the user and vital signs of the user to the baseline.

According to some embodiments determining a progress of the disease may include: identifying, based on comparing the characteristics to the reference set of characteristics, that a threshold was breached.

According to some embodiments the threshold may be defined based on at least one user associated parameter.

According to some embodiments, the at least one user associated parameter may be selected from a group consisting: a location of the user, an activity of the user, medical history of the user, and recent hospitalization information of the user.

According to some embodiments identifying an activity of the user may be based on input received from a component included in the system.

A system and a method according to some embodiments may include determining whether or not the user performed a physical activity prior to the determining of the progress of a disease; and if the user performed a physical activity prior to the determining of the progress of a disease then instructing the user to rest and breathe normally, recording of a respiration of the user, extracting one or more respiration characteristics from the recording, and determining a progress of the disease of the user based on comparing the one or more respiration characteristics to a reference set of characteristics.

According to some embodiments the processor may be further configured to: if the threshold was breached then: instructing the user to breathe normally; recording of a respiration of the user; extracting one or more respiration characteristics from the recording; and determining a progress of the disease of the user based on comparing the one or more respiration characteristics to a reference set of characteristics.

According to some embodiments the processor may be further configured to: determining nonadherence with a prescribed treatment based on at least one of: comparing respiratory characteristics of the user to a reference set of characteristics, and a report from an adherence system; and modifying the threshold according to a rule related to the user's medical condition and to the prescribed treatment.

According to some embodiments the processor may be further configured to calculate a biomarker score for the user based on comparing respiratory characteristics of the user to a reference set of respiratory characteristics.

According to some embodiments, the disease may be at least one of: asthma, COPD, pulmonary fibrosis, cystic fibrosis, bronchiectasis, interstitial lung diseases, heart failure and kidney failure.

### BRIEF DESCRIPTION OF THE DRAWINGS

Non-limiting examples of embodiments of the disclosure are described below with reference to figures attached hereto that are listed following this paragraph. Identical features that appear in more than one figure are generally labeled with a same label in all the figures in which they appear. A label labeling an icon representing a given feature of an embodiment of the disclosure in a figure may be used to reference the given feature. Dimensions of features shown in the figures are chosen for convenience and clarity of presentation and are not necessarily shown to scale.

The subject matter regarded as the invention is particularly pointed out and distinctly claimed in the concluding portion of the specification. The invention, however, both as to organization and method of operation, together with objects, features and advantages thereof, may best be understood by reference to the following detailed description when read with the accompanied drawings. Embodiments of the invention are illustrated by way of example and not limitation in the figures of the accompanying drawings, in which like reference numerals indicate corresponding, analogous or similar elements, and in which:

FIG. 1 shows high level block diagram of an exemplary computing device according to illustrative embodiments of the present invention;

FIG. 1B shows a user using an exemplary device according to illustrative embodiments of the present invention;

FIG. 2 shows a system according to illustrative embodiments of the present invention;

FIG. 3 shows an exemplary user computing device according to illustrative embodiments of the present invention;

FIG. 4 shows an exemplary respiration graph according to illustrative embodiments of the present invention;

FIG. 5 shows a flowchart of a method according to illustrative embodiments of the present invention;

FIG. 6 shows a flowchart of a method according to illustrative embodiments of the present invention;

FIG. 7 shows an exemplary screenshot according to illustrative embodiments of the present invention; and

FIG. 8 shows a flowchart of a method according to illustrative embodiments of the present invention.

It will be appreciated that for simplicity and clarity of illustration, elements shown in the figures have not necessarily been drawn accurately or to scale. For example, the dimensions of some of the elements may be exaggerated relative to other elements for clarity, or several physical components may be included in one functional block or element. Further, where considered appropriate, reference numerals may be repeated among the figures to indicate corresponding or analogous elements.

### DETAILED DESCRIPTION

In the following detailed description, numerous specific details are set forth in order to provide a thorough understanding of the invention. However, it will be understood by those skilled in the art that the present invention may be practiced without these specific details. In other instances, well-known methods, procedures, and components, modules, units and/or circuits have not been described in detail so as not to obscure the invention. Some features or elements described with respect to one embodiment may be combined with features or elements described with respect to other embodiments. For the sake of clarity, discussion of same or similar features or elements may not be repeated.

Although embodiments of the invention are not limited in this regard, discussions utilizing terms such as, for example, “processing,” “computing,” “calculating,” “determining,” “establishing”, “analyzing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium that may store instructions to perform operations and/or processes. Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. The term set when used herein may include one or more items. Unless explicitly stated, the method embodiments described herein are not constrained to a particular order or sequence. Additionally, some of the described method embodiments or elements thereof can occur or be performed simultaneously, at the same point in time, or concurrently.

As described, known systems and methods require a patient to actively participate in a process of monitoring and determining the medical condition of the patient and therefore depend on the patient adherence. For example, in order to obtain a measurement of a condition of a patient, known systems and methods typically require an action to be performed by a physician or by the patient, other systems and methods require a dedicated device to be attached to a patient. Therefore, known systems and methods do not enable continuous and/or early detection related to, for example, LD, COPD and/or other diseases, without interfering with the patient's activities and/or otherwise burdening the patient.

In contrast and as described, embodiments of the invention include a home monitoring system and method that doesn't require daily or other active participation of the patient. For example, embodiments of the invention enable automated, non-invasive characterization of LD and/or OLD, determining a progress or severity of LD and/or OLD, monitoring of aspects related to LD and/or OLD status over time, and early recognition of a LD and/or OLD for prompt institution of therapy where the characterization, monitoring and/or recognition are performed without requiring the patient (or a medical professional) to perform a specific activity. Moreover, in some embodiments, a process of monitoring, characterization and/or determination of a condition, state, trend, progress and/or improvement or deterioration of a user's medical condition may be performed without the user being aware of the process.

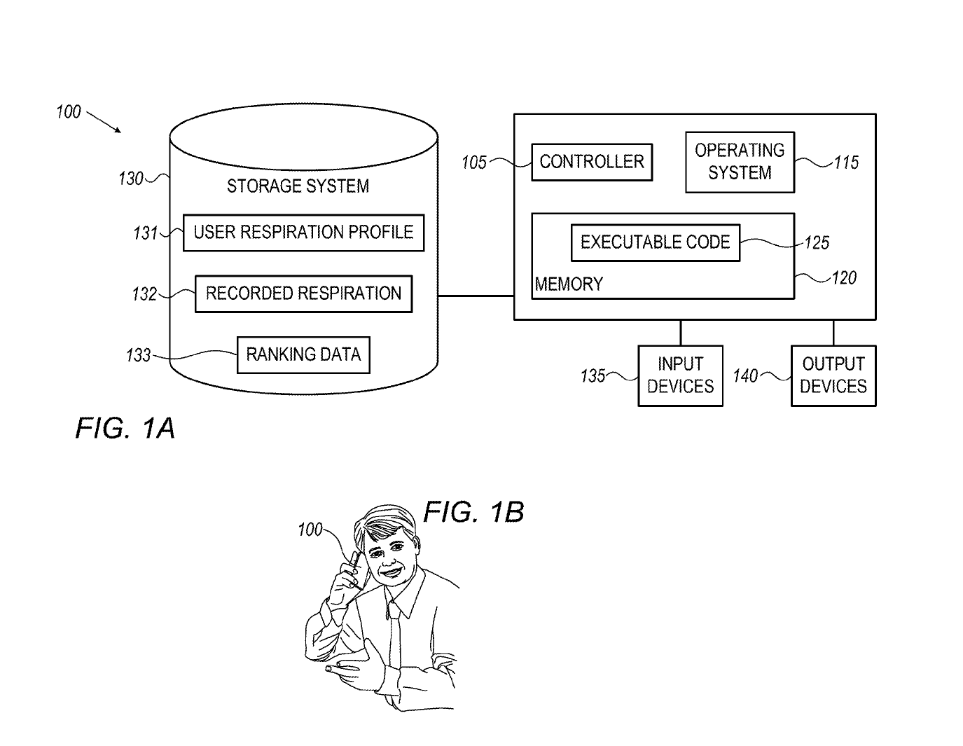

Reference is made to FIG. 1A, showing a high level block diagram of an exemplary computing device according to some embodiments of the present invention. In some embodiments, computing device 100 may be, or may be included in, a cellular telephone (e.g., smartphone or mobile phone as known in the art). Computing device 100 may include a controller 105 that may be, for example, a central processing unit processor (CPU), a chip or any suitable computing or computational device, an operating system (OS) 115, a memory 120, executable code 125, a storage system 130, input devices 135 and output devices 140. As shown, storage system 130 may include a user respiration profile 131, recorded respiration 132 and ranking data 133.

User respiration profile 131 may be a file or any other digital object as known in the art and may include values that represent respiration characteristics, e.g., a first value in user respiration profile 131 may be, or may represent, a depth (e.g., amount of air that is inhaled and exhaled), a second value in user respiration profile 131 may be, or may represent, a rhythm (e.g., a measure, or average of, an entire breathing cycle), a third value in user respiration profile 131 may be, or may represent, a length of pauses between breaths and other values in user respiration profile 131 may be, or may represent coughing, indication of sputum, amplitude, frequency, and the like. Recorded respiration 132 may be an audio file as known in the art, e.g., a file that include a digital representation of audio signals captured by a microphone as known in the art.

Controller 105 (or one or more controllers or processors, possibly across multiple units or devices) may be configured to carry out methods described herein, and/or to execute or act as the various modules, units, etc. More than one computing device 100 may be included in, and one or more computing devices 100 may be, or act as the components of, a system according to some embodiments of the invention.

OS 115 may be or may include any code segment (e.g., one similar to executable code 125 described herein) designed and/or configured to perform tasks involving coordination, scheduling, arbitration, supervising, controlling or otherwise managing operation of computing device 100, for example, scheduling execution of software programs or enabling software programs or other modules or units to communicate. OS 115 may be a commercial OS, e.g., OS 115 may be an Android or an iOS operating systems as known in the art.

Memory 120 may be or may include, for example, a Random Access Memory (RAM), a read only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short term memory unit, a long term memory unit, or other suitable memory units or storage units. Memory 120 may be or may include a plurality of, possibly different memory units. Memory 120 may be a computer or processor non-transitory readable medium, or a computer non-transitory storage medium, e.g., a RAM.

Executable code 125 may be any executable code, e.g., an application, a program, a process, task or script. Executable code 125 may be executed by controller 105 possibly under control of OS 115. For example, executable code 125 may be an application included in a communication device of, or operated by, a user (e.g., a smartphone, laptop or a home computer) that obtains information related to a user's medical condition, records, captures or otherwise obtains, while the user is using the communication device, respiratory characteristics of the user, and determines a condition, state, trend, progress and/or improvement or deterioration of the user's medical condition based on the user's medical condition and based on comparing the respiratory characteristics to a reference set of characteristics (or user respiration profile or reference respiration characteristics vector) as further described herein. The terms “user respiration profile”, “reference respiration characteristics vector”, “reference characteristics vector”, “reference vector” and “baseline” as referred to herein may mean, or relate to, the same thing or object. For example, any of a user respiration profile, reference respiration characteristics vector, reference characteristics vector, reference vector and/or baseline may include information that characterizes a user's medical condition, e.g., information such as respiratory parameters or values (e.g., rate, depth and so on), symptoms, medications and the like. Although, for the sake of clarity, a single item of executable code 125 is shown in FIG. 1A, a system according to some embodiments of the invention may include a plurality of executable code segments similar to executable code 125 that may be loaded into memory 120 and cause controller 105 to carry out methods described herein.

Storage system 130 may be or may include, for example, a flash memory, a universal serial bus (USB) device or other suitable removable and/or fixed storage unit. Content may be stored in storage system 130 and may be loaded from storage system 130 into memory 120 where it may be processed by controller 105. In some embodiments, some of the components shown in FIG. 1A may be omitted. For example, memory 120 may be a non-volatile memory (e.g., a flash memory in a smartphone as known in the art) having the storage capacity of storage system 130. Accordingly, although shown as a separate component, storage system 130 may be embedded or included in memory 120.

Input devices 135 may be or may include a microphone, a mouse, a keyboard, a touch screen or pad or any suitable input device. Input devices 135 may be, or may include, any system or device adapted to capture, obtain or measure signals related to a medical state of a user, for example, input devices 135 may include any one of: a stethoscope, a nasal pressure transducer, a CO2 sensor, a mercury strain gauge or sensors, a respiratory inductance plethysmography sensor, a Blood-Oxygen saturation (SpO2) sensor, a camera, a thermometer, an Electrocardiography (ECG) sensor, an Otoscope, a tongue depressor, a blood pressure monitor, a pulse oximetry sensing device, a Spirometer, a chemical test means and the like. It will be recognized that any suitable number of input devices may be operatively connected to computing device 100 as shown by block 135.

Output devices 140 may include one or more displays or monitors, speakers and/or any other suitable output devices. It will be recognized that any suitable number of output devices may be operatively connected to computing device 100 as shown by block 140. Any applicable input/output (I/O) devices may be connected to computing device 100 as shown by blocks 135 and 140. For example, a wired or wireless network interface card (NIC) or unit, a printer, a universal serial bus (USB) device or an external hard drive may be included in, or connected to computing device 100 as, input devices 135 and/or output devices 140.

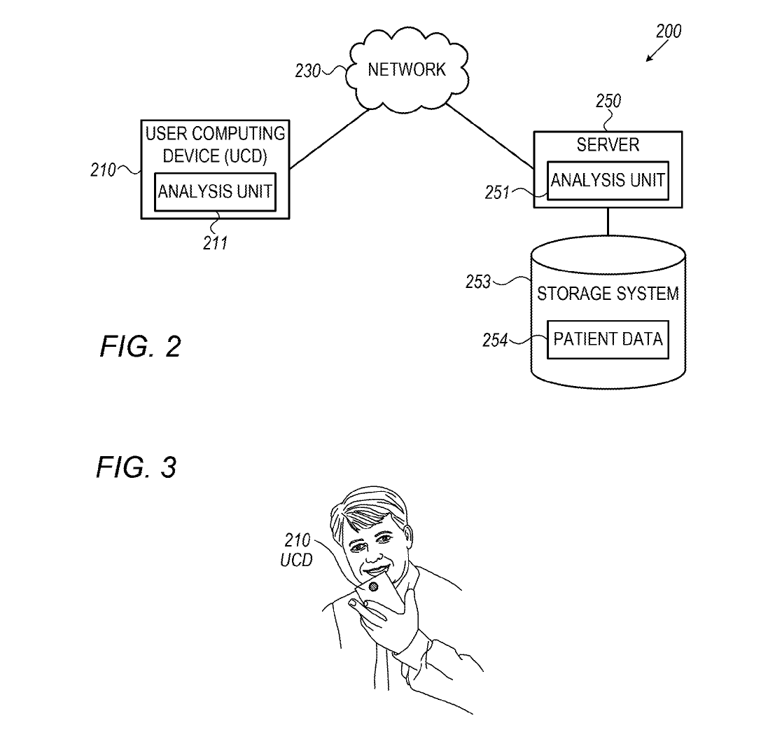

A system according to some embodiments of the invention may include components such as, but not limited to, a plurality of central processing units (CPU) or any other suitable multi-purpose or specific processors or controllers (e.g., controllers similar to controller 105), a plurality of input units, a plurality of output units, a plurality of memory units, and a plurality of storage units. A system may additionally include other suitable hardware components and/or software components. In some embodiments, a system may include or may be, for example, a personal computer, a desktop computer, a laptop computer, a workstation, a server computer, a network device, a tablet, a kiosk, a smartphone, a telephone, a smart watch, a wearable device, a medical device and any combination thereof, or any other suitable computing device. For example, a system as described herein may include one or more devices such as computing device 100.

Where applicable, units shown by FIG. 2 and other components and units described herein, may be similar to, or may include components of, device 100 described herein. For example, server 250 shown in FIG. 2 and further described herein may be or may include a controller 105, memory 120 and executable code 125. More than one computing device 100 may be included in a system, and one or more computing devices 100 may act as the various components of a system, for example, the components of system 200 such as user computing device (UCD) 210 and server 210 shown in FIG. 2.

As described, the present invention enables a home tele-monitoring system based on an algorithm designed to diagnose, determine a state, condition or progress of, and/or otherwise monitor diseases such as LD and OLD. As described, a system may record user respiration sounds and define or create a personalized respiration pattern or profile of the user. As further described, a system may determine a medical condition, state, trend, progress and/or deterioration based on comparing recorded user respiration sounds or other respiration characteristics of a user to a personalized respiration pattern or profile of the user.

An embodiment may provide an alert and/or feedback regarding a patient's condition and/or any development, state, status, deterioration or progress in the patient's physical and medical status to one or more of: the patient, a physician, a healthcare systems and/or any medical staff or institute connected to a system. An embodiment may record or capture a patient's (or user's) respiration sounds or characteristics during different time intervals, e.g., periodically or whenever a device is used, e.g., for a telephone conversation, for playing a game or for surfing the Internet. In some embodiments, a matrix or vector of parameters or values may be created by analyzing user respiration sounds and/or other data obtained by other sensors. An algorithm or logic used for analyzing user respiration sounds may be employed, e.g., by a user computing device and/or be a server.

An embodiment may analyze respiration sound patterns with respect to a profile, e.g., an embodiment may compare respiration sound patterns of a patient to respiration sound patterns obtained when the patient is in a reference, known and/or healthy state, accordingly, an embodiment may determine, identify and indicate a progress, trend, deterioration or improvement of a medical status or state of the patient. An embodiment may perform a dimensionality reduction of user respiration sounds or characteristics, e.g., a set of values calculated based on analysis of respiration of a user may be encrypted and included in a data vector. A vector as referred to herein may be a set, array, or sequence of values of a respective set, array, or sequence of parameters, e.g., a vector may be a set, array, or sequence of values of a frequency, amplitude and the like.

A database (e.g., in storage system 253 as described herein) may include personal medical file of a user, medical history, current medical data, allergies, chronic and medical treatment and the like. For example, patient data 254 described herein may include any medical and/or demographic data of a patient. For example, patient data 254 may include a physical symptom, physiological data, physical data, a medical history and use of medications. A server (e.g., server 250) may use data in a data base in order to analyze respiration sounds or respiration characteristics captured by a computing device as described and deduce or determine a condition, state, trend, progress or other aspect of a medical condition of a user.

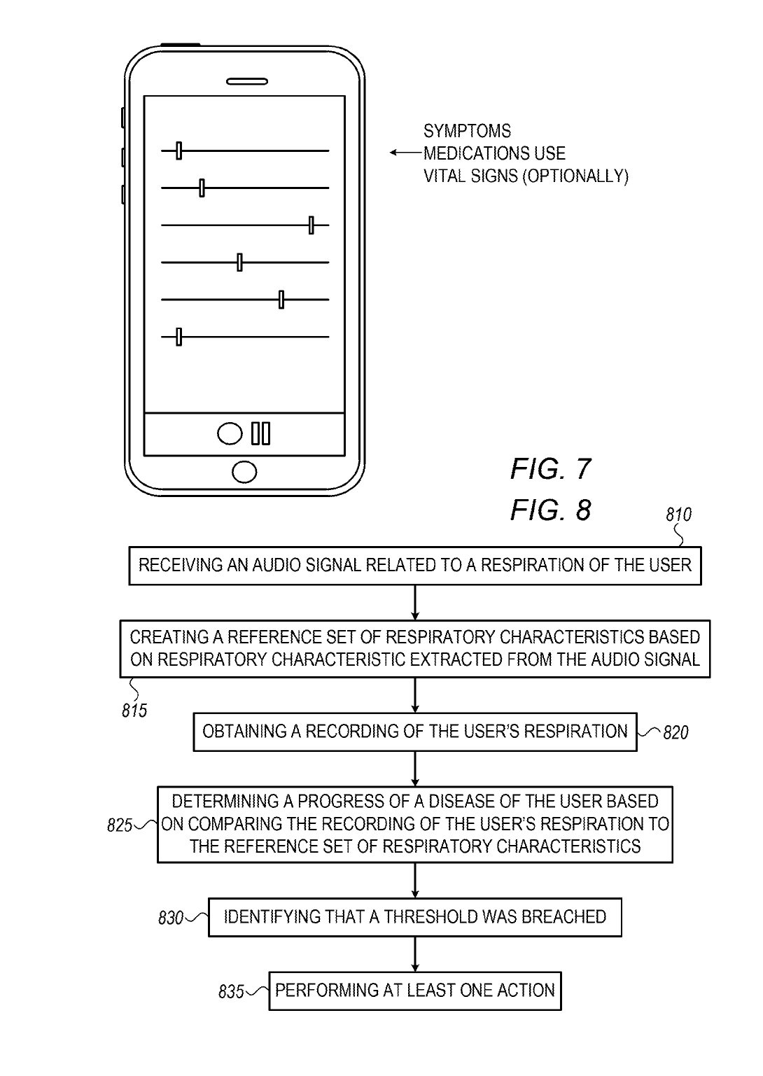

For example, physical symptoms included in patient data 254 may include an indication or a quantity of a breathing sound, breathing ratio, pain of any organ, fever, chills, infection, weakness, fainting, syncope, palpitations, dizziness, instability, nausea, vomiting, diarrhea, constipation, rash, pruritus, itching, swelling, lump, contusion, trauma, frequency/urgency (urine, stool), sputum type, color and/or production rate, chest tightness, cough, cough severity, dyspnea, wheezing, shortness of breath, bleeding, tiredness, confusion, restlessness, heart rate, tremor, sweating, edema, blurred vision, wound, fall, heartburn, burn, plegia, allergy, physiological feeling and a combination thereof. Medical data included in patient data 254 may include an indication, value, measure or a quantity related to respiratory sounds, respiratory airflow, respiratory related chest or abdominal movements, respiratory CO2 emission and oximetry, user's age, gender, race, emotional condition, physical condition, health condition and combination thereof. Other values, indications or information included in patient data 254 may be, or may be related to, for example, sputum amount, sputum color, breathing difficulty, dyspnea, speaking difficulty, physical activity intolerance, medications use, fever, wheezing, as well as medical history of the patient (e.g. recent hospitalizations, visits to emergency departments etc.). As described, data in patient data 254 may be automatically collected or calculated (e.g., based on recording breathing of a user and analyzing the recorded respiration), some of the data in patient data 254 may be provided by the user (e.g., using a screen as shown by FIG. 7), some of the data in patient data 254 may be received from a physician and yet other data in patient data 254 may be received from external systems, e.g., an ultrasound system and the like. Accordingly, it will be understood that the scope of the invention is not limited by the type or source of data in patient data 254.

As described, determining a severity or score may be based on patient data 254, for example, even if the respiration of two patients is similar or same, different medical conditions, severities or scores may be calculated or determined for the two patients based on their patient data 254, e.g., two users may have similar respiration characteristics, but a high score or severity may be calculated for the first user who suffers from asthma and a low score or severity may be determined for the second user who does not suffer from asthma. Any rule may be included, e.g., in ranking data 133, such that any data or information included in patient data 254 may be taken into account when calculating a score, severity, trend, improvement, deterioration or other aspects of a user's medical condition as described herein.

In some embodiments, a ranking scale or platform may be used in order to rank, score, or otherwise quantify a severity of a disease. For example, analysis unit 211 and/or analysis unit 251 may rank or score a state or progress of a disease (or otherwise determine a severity of the disease) based on rules, thresholds and criteria included in a ranking data 132 as described.

Determining a medical condition, e.g., identifying or determining a presence, state or progress of a disease of a user, may include or may be based on a score or rank that may be associated with, attributed to, or calculated for, a presence, state or progress of a disease of a user. Generally, a score or rank may be determined, calculated or quantified based on comparing a value in recorded respiration 132 to user respiration profile 131 such that a state or progress is measured or quantified by a rank or score. For example, if a breath rate of 5 is included or indicated in user respiration profile 131 and a breath rate of 8 is included or calculated based on data in recorded respiration 132 then a score of 3 may be determined A score as described may be calculated based on a number of parameters, e.g., a score may be calculated based on differences in values of rate, depth, rhythm, a length of pauses between breaths as measured or calculated based on, for example, subtracting values in recorded respiration 132 from the respective values in respiration profile 131. Different weights for different parameters or aspects may be included in ranking data 133 and may be used, for example, using a weight of 2 for respiration rate, a difference of 4 in the rate may contribute 8 to a score and using a weight of 0.5 for rhythm may cause a change of 3 in rhythm to add 1.5 to a score.

Different scores or ranks may be determined for different users, e.g., based on patient data 254. For example, even if the respiration characteristics of two patients are similar or same, different medical conditions, severities or scores may be calculated or determined for the two patients, e.g., based on their patient data 254, e.g., two users may have similar respiration characteristics, but a high score or severity may be calculated for the first user who suffers from COPD and a low score or severity may be determined, calculated or set for the second user who suffers from OLD. Any rule or threshold may be included, e.g., in ranking data 133, such that any data or information included in patient data 254 may be taken into account when calculating a score, severity, trend, improvement, deterioration or other aspects of a uses medical condition as described herein. In some embodiments, if a score or rank calculated as described is above a threshold score or value then an action may be performed, e.g., if a score calculated by analysis unit 211 as described is above a threshold then analysis unit 211 may generate an alarm, alert the user and/or a physician, send a message to server 250 or perform any action as described herein.

A score or rank, e.g., a severity score may be calculated, e.g., based on comparing data in recorded respiration 132 to user respiration profile 131 (e.g., based on rules or thresholds in ranking data 133 as described) a severity score may be calculated. For example, a first severity score may be calculated if a difference one respiration characteristics value (e.g., calculated by subtracting a value in recorded respiration 132 from a value in user respiration profile 131) is above or below a threshold, a second, higher severity score may be determined if differences of two respiration characteristics are above or below a respective two thresholds and so on. Of course, a severity score may be based on the magnitude of a breach of a threshold, e.g., a first severity score may be determined if a breath rhythm increases by 10% with respect to a previously measured breath rhythm or with respect to a breath rhythm in user respiration profile 131 and a second, higher severity score may be determined if the breath rhythm increases by 25%. A severity score may be sent to a server (e.g., analysis unit 211 may send a severity score to analysis unit 251) and, an alert or alarm may be generated and sent or provided if the severity score is above a predefined threshold, e.g., analysis unit 251 may send an alert message as described.

In some embodiments, based on comparing a respiration characteristics vector of a user to a reference respiration characteristics vector or to a user respiration profile, a severity or score may be determined for, or associated with, a medical condition of the user. An action may be performed based on the severity or score, e.g., a first severity or score may cause an embodiment to generate an alarm, a second (e.g., lower) severity or score may cause an embodiment to take another measure (or recording) of user's respiration and so on.

A ranking scale or platform may be used in order to rank, score or determine a severity of, a medical condition of a user. For example, analysis unit 211 and/or analysis unit 251 may rank, score or determine a severity based on rules and criteria included in a ranking definition (e.g., in ranking data 133) as described.

Any rule may be included, e.g., in ranking data 133, such that any data or information included in patient data 254 may be taken into account when calculating a score, severity, trend, improvement, deterioration or other aspects of a user's medical condition as described herein.

A set of thresholds, rules and/or criteria, e.g., included in ranking 133 may be used in order to determine a medical condition, e.g., in order to determine a state, progress or presence of a disease or a trend and/or an improvement or deterioration of a medical condition. For example, ranking data 133 may indicated that, if the length of pauses between words as determined or calculated based on recorded respiration 132 is greater than the length of pauses as included or indicated in user respiration profile 131 by more than 6 or by more than 20% than a deterioration or worsening of a disease is identified.

Any rule, criterion or threshold may be included in ranking data 133. For example, complex rules in ranking data 133 may include a breach of a number of thresholds related to a number of respiration characteristics. For example, a critical condition may be identified, by analysis unit 211 if a breath depth decreases by 15% and the length of pauses between breaths decreases by 10%. Thresholds, rules and/or criteria in ranking data 133 may be based on any information related to a user. For example, a first set of thresholds, rules and/or criteria may be used for a child, a second set of thresholds, rules and/or criteria may be used for an adult, a third set may be used for an elderly female and so on. Thresholds, rules and/or criteria in ranking data 133 may be automatically and/or dynamically modified. For example, if or when an alarming condition is identified as described, analysis unit 211 may automatically modify thresholds, rules and/or criteria in ranking data 133 such that values or changes in respiration characteristics that were previously regarded as normal (or of low severity) may now be regarded as indicating cause for alarm or high severity. For example, ranking data 133 may modified by analysis unit 211 or it may be downloaded, from or by, server 250, each time a change in the user's medical condition is identified or made known to system 200. For example, results from an ultrasound or other scan of a user may be provided to server 250 and, based on the results, ranking data 133 may be modified such that respiration characteristics previously regarded as normal may now be regarded as abnormal or indicating a severity above a threshold. Other causes for automatically modifying thresholds and rules in ranking data 133 may be a new prescription, new symptoms and/or any information relevant to a medical condition of a user.

Ranking of a respiration characteristics vector and/or determining that a threshold was breached may be based on patient data 254. For example, a deviation of 0.8 in the average length of pauses between breaths may be treated as cause for alarm if, as indicated patient data 254, the user is 80 years old and suffers from a known disease (e.g., OLD) in but may be regarded as normal (and therefore may not cause an embodiment to generate an alarm) if the patient or user is 45 years old.

Any other information related to a medical condition of a user or patient may be included in patient data 254 and may be used for determining a state or progress of a disease of the user. For example, thresholds used for identifying a state or progress of a disease, e.g., a deterioration of a medical condition or disease of a user may be set according to, or based on, medical information in patient data 254. For example, patient data 254 may include known (e.g., current, recent and/or historical) vital signs of a user (e.g., heart rate, blood pressure and the like), medications prescribed and/or used, symptoms the user has or suffers from, disease in the family, historical medical procedures or operations and the like. Thresholds used as described may be set or calculated based on patient data 254, for example, a first threshold for a minimal breath rate (or for an average pause between spoken words) may be set for a first patient if patient data 254 indicates that the patient is using a specific medication or that a specific surgery or other procedure was performed and a second threshold for a minimal pitch may be set for a second patient if patient data 254 of the second patient indicates other medications or surgeries.

Determining a presence, state or progress of a disease of a user or calculating a rank or score for a user may include identifying, detecting or determining any relevant aspect of a disease, for example, determining or identifying, by an embodiment, a presence of a disease may include identifying or detecting that a healthy user, e.g., one with no known medical history of a disease is now showing symptoms that may indicate the user has the disease, in other cases, determining or identifying, by an embodiment, a state, trend or progress of a disease may include identifying an improvement or worsening of, or related to, a disease.

Identifying or determining a state, trend or progress of a disease and/or calculating a score or rank as described may include identifying or determining and quantifying a trend or a rate of change. For example, based on repeatedly comparing respiration signals or aspects to reference respiration signals or aspects as described (e.g., over a number of days or weeks) the rate of improvement (or deterioration) of a disease may be determined and quantified.

For example, a change of ranks or scores calculated as described herein over time may be used in order to determine or quantify a trend, state or progress of a disease. For example, based on a change (over time) of a set of scores or ranks, an embodiment may determine how fast a disease is deteriorating or improving, accordingly, an effect or efficiency of a treatment may be measured or quantified. For example, after prescribing a specific medicine or other treatment for a disease, a physician may review reports from an embodiment that show or indicate a trend (e.g., an improvement or deterioration of the disease) and moreover, based on reports from a system (e.g., reports from server 250) that may include a rate of change as described, the physician may see or conclude the efficiency of the treatment based on the rate with which the patient is improving.

Determining, evaluating and quantifying (e.g., using scores or ranks as described) a condition, state, trend, progress and/or improvement or deterioration of a medical condition as described herein may include determining, evaluating and quantifying a rate of change of a medical condition, e.g., quantifying a rate of improvement. For example, the difference between scores calculated over a number of days may be used as an indication for a rate of change or a rate of improvement or deterioration of a medical condition. For example, a set of scores of 5, 10 and 15 calculated for 3 consecutive days may indicate (or be used by an embodiment to report) a rate of change of 5 and a set of scores of 3, 6, 9 may be used by an embodiment to determine a rate of change, a progress or a trend that is 3. Accordingly, an embodiment may provide a physician with a trend, progress and/or a measure of improvement or deterioration that is easily and/o intuitively understood by the physician. Moreover, providing a set of trends or progress indicators based on scores or ranks as described for a group of patients may enable a physician to quickly identify which patient in the group of patients is getting better or worse compared to other patients in the group, e.g., identify the patients in the group who respond well to a new drug or medication prescribed to the group.

An embodiment may provide feedback or indication regarding a current condition of the patient to the patient, to a physician, to a health care institute or to a member of a medical stuff. For example, server 250 may send alert messages, over a communication network, to a list or recipients as described herein.

Reference is made to FIG. 1B that shows a user using an exemplary device 100 according to illustrative embodiments of the present invention. As shown, device 100 may be, or may be included in, a smartphone (e.g., device 100 as shown by FIG. 1B may include a processor 105 and a memory 120). Accordingly, operations such as obtaining respiratory characteristics of a user, creating or generating a reference set of characteristics and determining a medical condition of the user based on comparing respiratory characteristics of the user to a reference set of respiratory characteristics may be performed in the background, while the user is using device 100 for various purposes (e.g., for playing games or for phone calls as shown by FIG. 1B), additionally, these operations may be performed without the user being aware that such operations are performed.

Reference is made to FIG. 2, an overview of a system 200 according to some embodiments of the present invention. As shown, a system 200 may include a UCD 210 that may include an analysis unit 211. Analysis unit 211 may be, or may include, a controller 105, a memory 120 and executable code 125 as described herein. As further shown, a system may include a server 250 that may include an analysis unit 251. Analysis unit 251 may be similar to analysis unit 211. As shown, server 250 may be operatively connected to a storage system 253 that may include or store patient data 254. As shown, a system 200 may include a network 230 that may enable server 250 and UCD 210 to communicate, e.g., exchange digital information as known in the art.

Network 230 may be, may comprise or may be part of a private or public IP network, or the internet, or a combination thereof. Additionally, or alternatively, network 230 may be, comprise or be part of a global system for mobile communications (GSM) network. For example, network 230 may include or comprise an IP network such as the internet, a GSM related network and any equipment for bridging or otherwise connecting such networks as known in the art. In addition, network 230 may be, may comprise or be part of an integrated services digital network (ISDN), a public switched telephone network (PSTN), a public or private data network, a local area network (LAN), a metropolitan area network (MAN), a wide area network (WAN), a wireline or wireless network, a local, regional, or global communication network, a satellite communication network, a cellular communication network, any combination of the preceding and/or any other suitable communication means. Accordingly, numerous elements of network 230 are implied but not shown, e.g., access points, base stations, communication satellites, global positioning system (GPS) satellites, routers, telephone switches, etc. It will be recognized that embodiments of the invention are not limited by the nature of network 230.

In some embodiments, UCD 210 may include, or may be connected to any sensor or measuring device system or equipment adapted to capture or obtain information or data that may be used in order to determine a condition, state, trend or progress related to a medical condition of the user. For example, I/O devices connected to UCD 210 may include a gyroscope, an accelerometer, a heart rate sensor, a temperature sensor, a GPS, a stethoscope, a nasal pressure transducer, a CO2 sensor, a mercury strain gauges, a respiratory inductance plethysmography, a Blood-Oxygen saturation (SpO2) sensor, a camera, a thermometer, an electrocardiography (ECG) sensing system, an Otoscope, a tongue depressor, a blood pressure monitor, a pulse oximetry, a Spirometer, a gas sensor, a pressure sensor, a chemical sensor. Server 250 may be connected to, or may obtain data from an Ultrasound system, a medical imaging system and the like. Accordingly, it will be understood that any medical information of a patient as known in the art may be available to a system and may be available and used when analyzing respiration data as described herein.

Reference is made to FIG. 3, showing an exemplary UCD 210 according to illustrative embodiments of the present invention. As shown, UCD 210 may be a smartphone that may be used as known in the art, e.g., for making telephone calls, playing games, chatting using instant messaging applications and the like. In some embodiments, when analysis unit 211 identifies or determines that the user is actively using UCD 210, analysis unit 211 may record user's breathing sounds or otherwise obtain respiratory characteristics of the user as described. Accordingly, obtaining respiratory characteristics of a user may be done automatically, possibly without a user being aware that a system is capturing (e.g., using a microphone in a smartphone or other computing device) and processing respiratory characteristics. As shown in FIG. 3, in some cases, a system and method may instruct a user to place UCD 210 on his or her throat or neck and the system or method may record respiratory characteristics of the user, e.g., using a microphone included in UCD 210. For example, if good quality signals cannot be captured by recording the user when he or she is using UCD 210 as described then a system or method may instruct the user to place UCD 210 as shown and may record the sound of the user's respiration. For example, at an initial or learning phase, an initial, good quality recording may be required and such good quality or reference recording may be obtained when UCD 210 is placed at an optimal location, e.g., on the user's throat as shown.

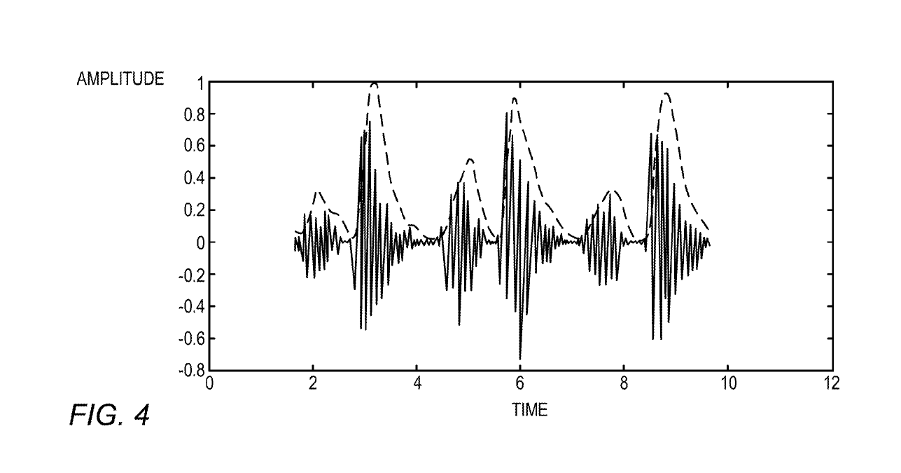

Reference is made to FIG. 4, an exemplary respiration graph according to illustrative embodiments of the present invention. As shown, a graph, trend or curve of a user's respiration may be captured or recorded and stored, e.g., as shown by user respiration profile 131 and/or recorded respiration 132. A graph, trend or curve as shown by FIG. 4 may be created or calculated based on recorded sounds or other data, e.g., based on recording a respiration of a user as described. It will be understood that the graph shown in FIG. 4 is presented for explanatory purpose and any representation of a respiration or respiration characteristics of a user may be used, e.g., by analysis unit 211 and/or by analysis unit 251. For example, based on data represented by the graph in FIG. 4, analysis unit 211 and/or by analysis unit 251 may determine respiration characteristics of a user such as rate, depth, rhythm, a length of pauses between breaths, coughing, indication of sputum, amplitude, frequency, and the like. Any relevant parameters or values may be extracted, by an embodiment, from a recorded breathing of a user. For example, analysis unit 211 may identify, extract or calculate, based on a recorded breathing (e.g., as shown by recorded respiration 132 and exemplified by FIG. 4) a respiratory rate, a wheezing, exhalation and inhalations times and ratio, coughing, pitch and/or mel-frequency cepstrum (MFC) coefficients as known in the art. As known in the art, MFC is a representation of power spectrum of a sound, a set of MFC coefficients (MFCCs) may be used in order to characterize sound.

Respiration characteristics of a user, e.g. included in a respiration characteristics vector, a reference respiration characteristics vector, or in a profile such as user respiration profile 131) may be, or may represent an inhalation, an exhalation, a breathing cycle, respiratory rate and/or breathing frequency curve over a time interval (e.g., in the form of sets of values representing, or usable for recreating, a time/frequency curve as known in the art).

As described, an embodiment may record a breathing sound of a user, e.g., recording may start automatically or upon a user's press on a record button (e.g., a graphical user interface (GUI) button presented, by analysis unit 211 on a screen of UCD 210). An embodiment may analyze or determine the quality of the recording. If the recording is of bad quality, an embodiment may notify the user and request, or instruct, the user to preform another recording. Obtaining a recording, determining its quality and obtaining another recording may repeated until a system identifies a good quality recording. The quality of a recording may be determined as known in the art, e.g., the quality of recorded sound may be determined, by analysis unit 211, based on the ability to extract respiratory characteristics from the recorded breathing, e.g., the ability to extract respiratory characteristics as included in user respiration profile 131 as described herein.

When a good quality recording is obtained, an embodiment may analyze the recording and determine respiratory characteristics, parameters, measures or values e.g., respiratory rate, expirum, inspirum, expirum/inspirum ratio, frequency and/or any respiratory characteristics as described herein, e.g., any respiratory characteristics included in user respiration profile 131 as described herein may be determined Based on respiratory characteristics determined based on a recorded respiration, an embodiment may determine a condition of a user, e.g., determine whether or not at least one respiratory characteristics value is abnormal, worse than a previously value, breaches a threshold and the like. If, based on a condition, state, trend or progress of a medical condition determined as described, a breach of a threshold is identified, a deterioration of a medical condition is determined or an undesirable trend is detected, an embodiment may perform one or more actions, e.g., generate an alarm as described.

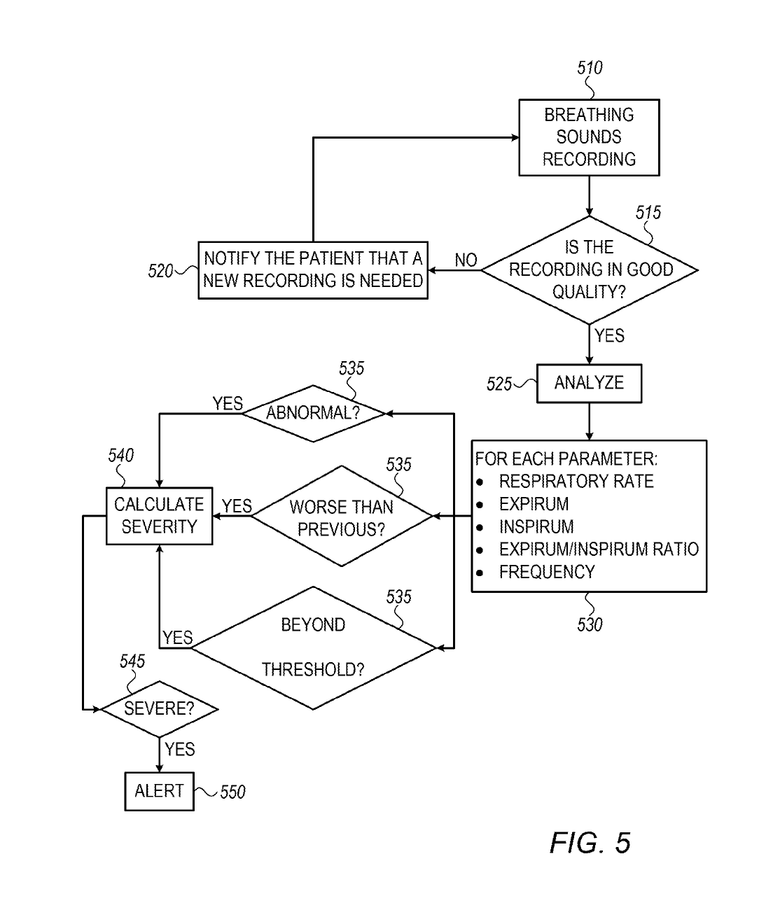

Reference is made to FIG. 5, a flowchart of a method according to illustrative embodiments of the present invention. As shown by block 510, an embodiment may record breathing sounds of a user, e.g., continuously, periodically and/or repeatedly, e.g., whenever a user is using UCD 210. Recorded respiration may be stored in recorded respiration 132. As shown by block 515, quality of a recorded breathing sound may be assessed as described herein and, as shown by block 520, if the recording is not usable for extracting or determining respiratory characteristics as described, an embodiment may notify the user that an additional recording is required, e.g., the user may be prompter, requested or instructed to record his or her breathing, e.g., press a GUI button as described. As shown by block 525, recorded breathing sounds may be analyzed, e.g., respiratory characteristics in recorded respiration 132 may be identified, determined and/or calculated as described herein. For example, and as shown by block 530, respiration rate, frequency, expirum, inspirum and a ration thereof may be calculated or determined based on recorded respiration sounds, e.g., by analysis unit 211.

As shown by blocks 535, an embodiment may determine a medical condition, a trend and/or an improvement or deterioration of a medical condition based on a recorded breathing sound. For example, a set of respiration characteristics may be extracted from, or determined or calculated based on recorded respiration 132 and the set of respiration characteristics may be compared to a set of respiration characteristics in user respiration profile 131. A set of thresholds, rules and/or criteria, e.g., included in ranking 133 may be used in order to determine a medical condition, a trend, state, progress and/or an improvement or deterioration of a medical condition. For example, ranking data 133 may indicated that, if a respiration rate as determined or calculated based on recorded respiration 132 is higher than the respiration rate in respiration profile 131 by more than 6 or by more than 20% than a deterioration or worsening of a medical condition is identified.

Any rule, criterion or threshold may be included in ranking data 133. For example, complex rules in ranking data 133 may include a breach of a number of thresholds related to a number of respiration characteristics. For example, a critical condition may be identified, by analysis unit 211 if a breathing rate increases by 15% and a breathing depth decreases by 10%. Thresholds, rules and/or criteria in ranking data 133 may be based on any information related to a user. For example, a first set of thresholds, rules and/or criteria may be used for a child, a second set of thresholds, rules and/or criteria may be used for an adult, a third set may be used for an elderly female and so on. Thresholds, rules and/or criteria in ranking data 133 may be automatically and/or dynamically modified. For example, if or when an alarming condition is identified as described, analysis unit 211 may automatically modify thresholds, rules and/or criteria in ranking data 133 such that values or changes in respiratory characteristics that were previously regarded as normal (or of low severity) may now be regarded as indicating cause for alarm or high severity. For example, ranking data 133 may modified by analysis unit 211 or it may be downloaded, from or by, server 250, each time a change in the user's medical condition is identified or made known to system 200. For example, results from an ultrasound or other scan of a user may be provided to server 250 and, based on the results, ranking data 133 may be modified such that respiratory characteristics previously regarded as normal may now be regarded as abnormal or indicating a severity above a threshold.

As shown by block 540, a severity may be calculated, e.g., based on comparing recorded breathing or respiration 132 to user respiration profile 131 (e.g., based on rules or thresholds in ranking data 133 as described) a severity score may be calculated. For example, a first severity score may be calculated if one respiration value is above or below a threshold, a second, higher severity score may be determined if two respiration values are above or below a respective two thresholds and so on. Of course, a severity score may be based on the magnitude of a breach of a threshold, e.g., a first severity score may be determined if a respiration rate increases by 10% with respect to a previously measured respiration rate or with respect to a respiration rate in user respiration profile 131 and a second, higher severity score may be determined if the respiration rate increases by 25%. As shown by block 545, a severity score may be sent to a server (e.g., analysis unit 211 may send a severity score to analysis unit 251) and, as shown by block 550, an alert or alarm may be generated and sent or provided, e.g., analysis unit 251 may send an alert message as described.

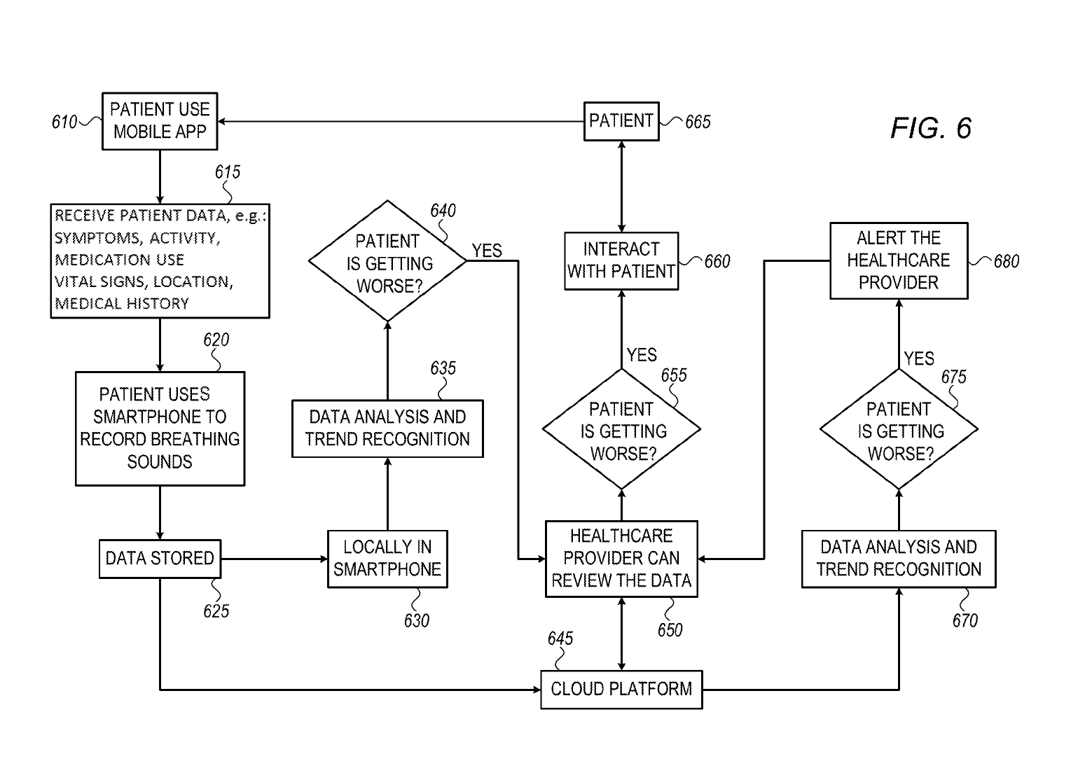

Reference is made to FIG. 6, a flowchart of a method according to illustrative embodiments of the present invention. As shown by block 610, an embodiment may identify that a user is using UCD 210. For example, when a user switches UCD 210 on, presses a button (e.g., the home button of a smartphone), launches an application (e.g., a game or a chat application) or otherwise interacts with UCD 210, analysis unit 211 may be notified (e.g., by OS 115 as described) and may start recording user's respiration sound.

As shown by block 615, a user may be prompted to enter or provide information. For example, analysis unit 211 may present, on a screen of UCD 210, a form that may be used by a user in order to provide any relevant information or data. For example, any information related to a medical condition such as, vital signs (e.g., heart rate, blood pressure and the like), medications prescribed and/or used, symptoms the user has or suffers from, disease in the family, historical medical procedures or operations, recent hospitalizations, visits to emergency units, and the like may all be entered and save, e.g., in patient data 254. For example, any information provided by a user may be sent, by analysis unit 211 to server 250 and may be stored in a user profile, e.g., in patient data 254. According to some embodiments, patient data 254 may further include user associated data such as location, activity level and the like received from UCD 210. According to some embodiments, UCD 210 may measure the patient daily activity, e.g., how many steps, walking speed, hours of daily activity, heart rate during activity and other physiological measurements, such as number of breaths per minute. Any reduction of the measured daily activity, may be related to, or may indicate a worsening in the patient's disease.

Reference is additionally made to FIG. 7 that shows an exemplary screenshot according to some embodiments of the invention. As shown in FIG. 7, slide bars may be used in order to easily enter information, e.g., as known in the art. For example, using a screen as shown by FIG. 7, a user may enter and age, gender, vital signs, medications used and the like and the information provided by the user may be stored, e.g., in a user profile and/or patient data, e.g., in patient data 254.

As shown in FIG. 7, information related to, or indicating medications, vital signs and symptoms may be provided, by a user to a system. For example, an embodiment may periodically (e.g., once a day or once a week) prompt a user (e.g., by presenting a screen or questionnaire as shown by FIG. 7) to provide, inform, enter or indicate the user's vital signs (e.g., heart rate or blood pressure that may be measured using any system or method as known in the art). An embodiment may periodically prompt a user to indicate which medications are used by the user e.g., type and dosage of pills or other medications. An embodiment may periodically prompt a user to report symptoms, e.g., specific pains, allergies, skin rash and the like.

An embodiment may use information related to medications, vital signs and symptoms for ranking a medical condition of a user, e.g., in addition to comparing respiratory characteristics of the user to a baseline or reference respiratory characteristics of the user. For example, thresholds used as described may be set, determined or adjusted based on medications consumed by a user and/or based or according to the user's vital signs and/or based on symptoms. For example, a threshold related to an increase in breathing rate may be set to 10% (e.g., an increase of more than 10% may cause an embodiment to take action, e.g., raise or send an alarm) and, if chest pain (a symptom) is reported by the patient, the threshold may be decreased to 5%, or the threshold may be decreased if the heart rate (a vital sign) of the patient increases by 12% or the threshold may be modified if the patient starts taking a specific pill or medicine.

A baseline or a user's respiration profile (or reference respiration characteristics vector, reference characteristics vector or simply a reference vector) may be created and/or updated by continuously, periodically or iteratively obtaining a recording of user respiration, user associated data (such as medical history, user location, user activity and the like) and additional data (e.g., vital signs, medications and symptoms) and used for determining a progress of a condition, e.g., a progress or trend of an OLD as described.

For example, during an exacerbation, a patients may cough more, have more breathing difficulty and so on, accordingly, by comparing the rate, depth or frequency of coughing of a user to a baseline (that may be include the rate of coughing as identified in the past) the medical condition of the user may be defined, similarly, by identifying a change in a breathing pattern and/or symptoms (e.g., by comparing recently measured breathing pattern and/or symptoms to past breathing pattern and/or symptoms in a baseline or profile), a progress (e.g., deterioration or improvement) of a medical condition may be determined In some embodiments, evaluating a large number of parameters as described may be done using artificial neural network (ANN) techniques as known in the art. For example, ANN may be used for comparing a set of respiration characteristics values, vital signs values, symptoms indications and medication dosages in a baseline or profile created on a first day to a respective set of values obtained or determined in the following day.

As shown by block 620, a smartphone may be used in order to record patient's breathing sounds, e.g., as described herein. As shown by block 625, recorded breathing may be stored, e.g., in recorded respiration 132 as described herein. As shown by block 645, data recorded may be sent to a cloud platform, e.g., to server 250 as described herein. As shown by block 630, various operations may be performed locally, e.g., on or by UCD 210. For example, as shown by block 635 data analysis and/or trend recognition may be performed locally, e.g., by analysis unit 211 that may be local, e.g., included in UCD 210 as described. As shown by block 640, it may be determined locally, e.g., on or by a smartphone carried and/or used by a user, that the condition of the user is worsening, deteriorating or requiring attention, e.g., analysis unit may determine that the user's medical condition is such that an alarm or attention are required as described.

As shown by block 650, data sent to a cloud platform may be provided to and/or reviewed by, a healthcare provider, e.g., a physician. For example, server 250 may identify a deterioration of a medical condition based on analysis of breathing of a patient as described and may send data to a physician for inspection. For example, a patient profile stored in storage system 253 may include a name, telephone number, an email and/or any other information related to a physician who is treating the patient and, upon detecting a deterioration in the medical condition of the patient, server 250 may send a message to the physician, e.g., an email or a Short Message Service (SMS) or text message as known in the art.

As shown by block 660, if a deterioration in the medical condition of a patient is detected, an embodiment may interact with a patient 665, e.g., using the patient's smartphone, an embodiment may instruct the patient to lie down and rest, call an ambulance or perform any other operation. An embodiment may instruct a patient 665 to record his or her breathing as described herein, e.g., in order to determine whether a deterioration in medical condition identified is erroneous (e.g., a false positive as known in the art). Accordingly, based on an identified condition, state, trend, progress or other aspect of a medical condition, an embodiment may interact with a patient and provide the patient with instructions designed to best handle a medical emergency and/or acquire additional medical readings or measurements of the patient.

As shown by block 670, a cloud platform may perform analysis of recorded breathing, e.g., server 250 may analyze recorded breathing provided by UCD 210 as described herein. As shown by block 675, if it is determined that the medical condition of a patient or user is getting worse, a healthcare provider may be alerted by the cloud platform, e.g., by server 250 as described herein.

As described, an embodiment may determine whether or not a user is operating or using UCD 210 and, upon determining that UCD 210 is being used, an embodiment may record respiration sounds as described. For example, an embodiment may repeatedly or periodically, or based on an event, activate a microphone in UCD 210 and record breathing or respiration sounds of the user (or record other respiration characteristics using any sensor) and analyze the recorded breathing as described. Accordingly, an embodiment may continuously or periodically monitor and/or determine a user's medical condition, state, trend and/or progress.

As described, an embodiment may prompt a user to breath normally for an indicated time period, and may record user's respiration. For example, if analysis unit 211 determines that user's respiration has not be obtained or captured for more than a predefined period of time (e.g., one day) then analysis unit 211 may prompt the user to breath normally and may record user's respiration as described. In some embodiments, e.g., in order to create a baseline profile, analysis unit 211 may instruct a user to assume a specific position (e.g., sit in chair or lie down) and guide the user to breath normally until a clear, good quality audio signal of the user's respiration is captured.

As described, a respiration characteristics vector may be produced, generated or created based on analysis of user respiration. For example, a profile or a respiration characteristics vector may include values or representations of respiration, e.g., values or representations of breath rate, depth and/or rhythm, a length of pauses between breaths, coughing, indication of sputum, amplitude, frequency, and the like.

A respiration profile (e.g., user respiration profile 131) may include a specific set of values for a predefined set of parameters as derived or determined based on respiration of a user. A respiration profile (e.g., user respiration profile 131) may be created based on normal respiration of the user, e.g., when the medical condition of the user is known, and, accordingly, user respiration profile 131 may represent the user and may be used in order to assess or determine the user's medical condition, state, trend or progress as described herein. A respiration characteristics vector may be, or may include values or representations of respiration e.g., as described with respect to user respiration profile 131. For example, user respiration profile 131 may describe or represent a user as described and a respiration characteristics vector may be a temporal, realtime or current representation of the user's respiration.

Analysis unit 211 may perform any action based on a classification or examination of recorded respiration. For example, based on comparing a respiration characteristics vector representing a current condition of a user to a user respiration profile 131 that represents a normal (or known) condition of the user and determining a breach of at least one threshold, analysis unit 211 may: send a message to a predefined list of recipients (e.g., a physician of the patient, a medical institution, a family relative and the like); sound an alarm (e.g., using a speaker in UCD 210), present a warning message (e.g., using a display of UCD 210) or perform any other action. An alarm generated by UCD 210 may be or may include, for example, a presentation of text and/or an image on a display of UCD 210, using a speaker of UCD 210 to sound an alarm, using a vibration unit included in UCD 210 to vibrate UCD 210 and the like.

In some embodiments, if, based on comparing a respiration characteristics vector representing a current condition of a user to a user respiration profile 131 analysis unit 211 determines or identifies a deviation of one or more characteristics is above a threshold then analysis unit 211 may prompt the user to breath normally, e.g., while sitting up straight or lying down, may capture user's respiration as described, create (or recreate) a respiration characteristics vector and verify that conditions for alarm indeed exist by comparing the newly created respiration characteristics vector to the user's respiration profile, accordingly, false alarms may be avoided.

A respiration characteristics vector created as described may be sent or uploaded to a server, e.g., analysis unit 211 may send or upload a respiration characteristics vector to server 250 where analysis unit 251 may analyze the uploaded respiration characteristics vector, e.g., as described with reference to analysis unit 211.

Server 250 may analyze, examine or process a respiration characteristics vector based on any data or information related to a user or patient, e.g., data included in patient data 254. For example, patient data 254 may include medical history and condition and/or demographic information of a user of UCD 210 and server 250 may analyze a received respiration characteristics vector based on such data. Ranking of a respiration characteristics vector and/or determining that a threshold was breached may be based on patient data 254. For example, a deviation of 0.8 in average pause may be treated as cause for alarm if the user is 80 years old and suffers from a known disease (e.g., OLD) but may be regarded as normal (and therefore may not cause an embodiment to generate an alarm) if the patient or user is 45 years old.

As described, an embodiment (e.g., controller 105 included in analysis unit 211) may receive an audio or other signal related to a user's respiration and may determine a medical condition, state, trend and/or progress of the user based on comparing the signal to a reference signal. For example, UCD 210 may be a mobile communication device (e.g., a smartphone) owned and/or operated by a user or patient and, when a user uses UCD 210 for a phone call, analysis unit 211 may automatically record the user's respiration. By automatically, in the background and without user intervention, awareness or effort, obtaining user's respiration characteristics as described, processing the obtained respiration characteristics as described, determining a medical condition or progress and, if need be, alerting as described, a system and method may provide or enable advantages over known or existing systems and methods. The advantages of an automated system and method that continuously, periodically and/or repeatedly monitors and determines a user's medical condition, state, trend and/or progress without burdening the user or patient and/or a medical professional may be appreciated by a person skilled in the art.

As described, a trend or progress of a medical condition, e.g., a deterioration or improvement, may be determined based on comparing temporal, current or realtime respiration characteristics of a user with a reference set of respiration characteristics (e.g., included in a profile as described).

Comparing vectors may be done as known in the art. For example, relating or comparing vectors (e.g., comparing a current, new or recently generated respiration characteristics vector with or to a baseline respiration characteristics vector or a vector in a profile) may include defining and using a space of interest. Otherwise described, relating or comparing vectors may include relating or comparing the projections of the vectors on a predefined space.

For example, distance between a respiration characteristics vector and a reference respiration characteristics vector in a predefined space (e.g., a space defined by coordinates that are respiration rate and respiration depth) may be calculated or determined as known in the art and the distance may be compared to a threshold or predefined value. In some embodiments, if the distance between a respiration characteristics vector and a reference respiration characteristics vector is above a threshold, the embodiment may perform an action as described, e.g., generate and send an alarm message, display a warning on a display of UCD 210, send an email to a physician and so on.

It will be understood that any space of any dimension for comparing vectors may be defined and, accordingly, that vectors of any dimension may be compared or matched. Accordingly, the dimensionality or space used for vector operations may be reduced. For example, if it is known that OLD or progress thereof may be identified based on two aspects of user's respiration (e.g., rate and depth) then an embodiment may use a two-dimension vectors and space in order to evaluate, determine or assess a condition or progress of OLD of a user.

Reference is made to FIG. 8, a flowchart of a method according to illustrative embodiments of the present invention. As shown by block 810, an audio signal related to a user's respiration may be received. For example, analysis unit 211 may receive, from a microphone included in UCD 210, an audio signal that is the sound of a user's breath as described. As shown by block 815, a reference set of respiratory characteristics may be created based on respiratory characteristics extracted from the audio signal.

For example, analysis unit 211 may create a reference set of respiratory characteristics based on recording normal respiration of the user as described. A reference set of respiratory characteristics may be continuously, periodically or iteratively updated, e.g., by periodically recording respiration of the user during telephone calls made by the user or when the user is using UCD 210 and updating the reference set of respiratory characteristics, for example, recorded respiration 132 may be used in order to update user respiration profile 131 (e.g., by a reference set of respiratory characteristics in user respiration profile 131) such that user respiration profile 131 is kept up-to-date and reflects the user's current state, e.g., reflects or represents the user's medical condition in terms of respiration sounds or characteristics.

In some embodiments, a reference set of respiratory characteristics and/or user respiration profile 131 may be created by instructing the user to breathe normally, recording the user's respiration sound and creating the reference set of respiratory characteristics and/or user respiration profile 131 based on the recording and/or based on respiration sounds or characteristics extracted from the recording. For example, a reference set of respiratory characteristics and/or user respiration profile 131 may include sounds, values, indications or characteristics of rate, rhythm, a length of pauses between breaths, an amount or depth (e.g., amount of air that is inhaled and exhaled) and the like.

As shown by block 820, a recording of the user's respiration may be obtained. For example, after a reference set of respiratory characteristics and/or user respiration profile 131 (or a baseline as described herein) is created, an embodiment may record the user's breath as described herein.

As shown by block 825, a progress of a disease may be determined based on comparing the recording of the user's respiration to a reference set of respiratory characteristics and/or user respiration profile 131. For example, analysis unit 211 may compare a new or recently recording of a respiration of a user to a reference set of respiratory characteristics (e.g., included in user respiration profile 131) and determine a presence, state or progress of a disease based on differences found between the newly or recently recorded respiration and a reference set of respiratory characteristics, user respiration profile 131, a baseline or a profile of the user as described.

As shown by block 830, a breach of a threshold may be identified. For example, a breach of one or more thresholds related to variations or differences of respiration (e.g., differences between a characteristic in a reference respiratory and a characteristic extracted from a recoded respiration) may be determined or identified by comparing the characteristics in a recorded respiration to the characteristics in a profile (e.g., user respiration profile 131) and/or to the characteristics in a baseline or in a reference set of respiratory characteristics, e.g., by analysis unit 211 as described.

As shown by block 835, at least one action may be performed. For example, if analysis unit 211 identifies that a threshold was breached or a calculated score is greater than a predefined value then analysis unit 211 may generate an alarm or alert as described.

In some embodiments, thresholds may be dynamically modified based on various inputs, aspects or considerations. For example, a threshold included in ranking data 133 as described may be defined, set or updated based on a location of the user or the location of UCD 210. For example, analysis unit may receive a location of UCD 210 from a GPS unit included in UCD 210 and may alter or modify a threshold based on the location. For example, as known in the art, respiration (e.g., rate, rhythm, length of pauses between breaths and the like) may vary based on altitude, pollution, weather and the like. For example, if it is determined, based on a location, that the user is at high altitude, analysis unit 211 may change a threshold related to rate, rhythm or length of pauses between breaths such that the variation of the user's respiration caused by the high altitude is taken into account, e.g., the threshold value may be increased to accommodate a shift in breath rate that may be caused by thinner air in high altitudes.

In embodiments, analysis unit 211 may report a location of UCD 210 to server 250 and receive, from server 250, information relevant to the location, e.g., weather conditions, level of pollution, dust or smoke levels in the area and the like. As described with reference to altitude, analysis unit 211 may dynamically modify thresholds, criteria or logic, e.g., included in ranking data 133 such that aspects such as weather conditions, level of pollution and the like are taken into account when determining a progress or severity of a disease as described, e.g., by changing thresholds as described.

In some embodiments, a threshold (e.g., included in ranking data 133 as described) may be defined, set or updated based on an activity of the user. For example, as known in the art, a person's respiration (e.g., rate, rhythm and/or depth) may vary when physically strained.

In some embodiments, analysis unit 211 may determine or identify an activity of a user, e.g., based on input from an accelerometer unit (or a gyroscope unit) included in UCD 210, for example, analysis unit 211 may identify or determine that the user is now running or walking fast based on a built-in accelerometer gyroscope unit, e.g., an accelerometer gyroscope unit included in UCD 210 as known in the art. Based on an activity of the user, analysis unit 211 may dynamically modify or change thresholds, for example, if it is determined that the user is running then thresholds related to breath rate or depth, pauses and/or frequency may be changed in order to accommodate natural changes or shifts in respiration characteristics.

In some embodiments, if analysis unit 211 determines whether or not the user of UCD 210 performed a physical activity prior to the determining of the progress of a disease and, if the user performed a physical activity (possibly immediately) prior to the determining of the progress of a disease as described then analysis unit 211 may instruct the user to rest or lie down or sit. Either after a user confirms performing an instruction such as resting or after a predefined interval has passed since the physical activity was performed, analysis unit 211 may instruct, request or otherwise cause, the user to breathe normally (e.g., as described herein) and may reevaluate the user's medical condition, e.g., determine a state or progress of a disease by recording of a respiration of the user, extracting one or more respiration characteristics from the recording; and determining a progress of the disease of the user based on comparing the one or more respiration characteristics to a reference set of characteristics. Accordingly, false positives as known in the art may be avoided.

In some embodiments, if analysis unit 211 determines that a threshold was breached as described herein then analysis unit 211 may reevaluate the user's medical condition, e.g., determine a state or progress of a disease by recording of a respiration of the user, extracting one or more respiration characteristics from the recording; and determining a progress of the disease of the user based on comparing the one or more respiration characteristics to a reference set of characteristics. Accordingly, false positives as known in the art may be avoided. Accordingly, false positives, e.g., related to a wrongly identified threshold breach, may be avoided.

An embodiment may determine nonadherence with a prescribed treatment based on at least one of: comparing a set of respiratory characteristics (or a recorded respiration) to a reference set of respiratory characteristics and/or to a user respiration profile 131 and an embodiment may record and report nonadherence. For example, analysis unit 211 may receive reports from a system that records or measures dosages of medicine (e.g., a system that tracks medication use as known in the art) and, based on the reports, determine whether or not the user is taking his or her pills at the right times and dosages, uses an inhaler and so on. Other devices or systems that may be operatively connected (e.g., over a Bluetooth, WiFi or other network) to analysis unit 211 and report adherence may be an oxygen saturation metering system, a peak flow meter, a spirometer and so on.

If nonadherence is determined or identified as described, analysis unit 211 may remind the user to use medications or adhere to a treatment (e.g., using a screen or speaker of UCD 210 as described). If nonadherence is determined or identified as described, analysis unit 211 may change thresholds or criteria such that nonadherence and its effect are taken into account when determining a state or progress of a disease. For example, if it is known that a patient who suffers from a specific disease and who, in addition, is not taking his or her medicine, is at a higher risk then a threshold may be lowered such that an alarm that would not be generated when (or if) the user takes his or her medicine as prescribed will now be generated as described. For example, a rule may be specific to the user's medical condition and to the prescribed treatment (and possibly to a specific threshold) and, upon determining nonadherence as described, the rule may be used in order to modify the threshold.

Various methods may be used in order to identify or determine nonadherence. For example, if an improvement in medical condition of a user is expected in light of a new prescription (that may be known to analysis unit 211, e.g., based on information received from server 250 or based on input from a user) but no improvement is identified during an indicated time period then analysis unit 211 may determine that the user does not adhere to the new prescription. For example, analysis unit 211 may receive a message from server 250 that, e.g., based on patient data 254, informs analysis unit 211 that the user is now expected to take a new pill and that an improvement is expected inside one week. In such case, if analysis unit 211 does not identify an improvement within a week, analysis unit 211 may determine nonadherence, e.g., determine that the user does not take his pills as prescribed.

For example, if medication A is taken 3 times a day, and changes are detected in the breathing pattern in the morning and the evening but not at noon, it may be assumed that the patient is skipping his noon dose.

Accordingly, an embodiment may accurately determine a medical condition and/or accurately identify a state or progress of an illness or disease even under changing and/or different conditions, in different locations, when medications are changed and so on. Moreover, false alarms (e.g., false positives) may be avoided by, when determining a state or progress of a disease or otherwise evaluating a medical condition as described, taking into consideration or account a location, an activity, an altitude, a weather and other aspects as described. As described above, activity level of a patient may be monitored to identify changes in activity levels indicative of a worsening in a medical condition of the patient.

In some embodiments, a biomarker score may be calculated for a user by comparing respiratory characteristics of the user (e.g., characteristics in, determined based on, or extracted from, recorded respiration 132 as described) to a reference set of respiratory characteristics, e.g., a reference set of respiratory characteristics included in user respiration profile 131.

For example, by comparing recorded respiration 132 (or characteristics extracted therefrom) to user respiration profile 131 and/or by respiration analysis as known in the art, a biomarker score that indicates or quantifies a breathing difficulty of the user may be produced. In other embodiments, a biomarker score may be used for evaluation in an emergency department or in a clinic visit, or for monitoring progress during hospitalization or clinical trials.

As described, recorded respiration may be uploaded to a server (e.g., to server 250) and the server may determine a medical condition of the user based on comparing the recorded respiration to a reference respiration and based on any medical or other information related to the user. For example, examining or comparing vectors as described may be based on a user's medical record or demographic data. For example, an embodiment may generate an alarm if a distance between a respiration characteristics vector and a reference respiration characteristics vector is greater than a threshold. In some embodiments, the threshold may be user specific, and/or dynamically set. For example, in some embodiments, a first threshold related to vector distance as described may be used for a patient who suffers from OLD, a second threshold may be used for a patient who suffers from COPD, a third threshold may be used for a patient with Asthma and so on. In another aspect, a first threshold related to vector distance as described may be used for a patient who is a 45 years old female, a second threshold may be used for a patient who a 45 years old male, a third threshold may be used for children and so on.

As described, an embodiment may include a system and method for diagnosing, identifying or determining a condition, state or a progress of related to, LD, e.g., OLD. For example, a system may include a communication device that includes a memory and a processor or controller (e.g., controller 105) and the processor or controller may be configured to obtain information related to a user's medical condition, obtain, while the user is using the communication device, respiratory characteristics of the user and determine a medical condition of the user based on the user's medical condition and based on comparing the characteristics to a reference set of characteristics. For example, controller 105 included in UCD 210 may obtain information related to a user's medical condition (e.g., provided by the user as described or downloaded from server 250), obtain respiratory characteristics of the user by recording user's respiration as described and determine a medical condition of the user by comparing or relating obtained respiratory characteristics of the user to a profile, baseline or reference set of respiratory characteristics, e.g., in a respiration profile as described. As described, a reference set of characteristics may be created, e.g., by controller 105, by obtaining signals related to the user's breathing while the user uses the communication device. As described, a reference set of characteristics (e.g., included in a profile as described) may be created based on the characteristics of a normal breathing of the user.

As described, based on comparing a breathing or respiration of a user (e.g., as represented by a recorded breathing as described) to a reference respiration vector or to a respiration user profile, a severity or score may be determined for, or associated with, a medical condition of the user. As described, an action may be performed based on the severity or score, e.g., a first severity or score may cause an embodiment to generate an alarm, a second (e.g., lower) severity or score may cause an embodiment to take another measure (or recording) of user's breath and so on.

As described, a ranking scale or platform may be used in order to rank, score or determine a severity of a medical condition of a user. For example, analysis unit 211 and/or analysis unit 251 may rank, score or determine a severity based on rules and criteria included in a ranking definition as described.

As described, embodiments of the invention improve the field of monitoring patients, e.g., by enabling a system and method that continuously, periodically or repeatedly monitors and determines a medical condition without requiring dedicated equipment to be attached to a patient and further without burdening the patient. In another aspect, embodiments of the invention improve the field of monitoring patients by enabling remote OLD monitoring using a home, or a user device for monitoring and/or determining a state, condition or progress of, OLD as described.

Embodiments of the invention address the computer-centric challenge of computerized monitoring related to health or medical condition. Unlike known systems and methods that require and use dedicated devices or systems, embodiments of the invention address the computer-centric challenge of computerized health monitoring and alerting using a device that is normally carried and operated by a user (e.g., using a smartphone as described).

In the description and claims of the present application, each of the verbs, “comprise” “include” and “have”, and conjugates thereof, are used to indicate that the object or objects of the verb are not necessarily a complete listing of components, elements or parts of the subject or subjects of the verb. Unless otherwise stated, adjectives such as “substantially” and “about” modifying a condition or relationship characteristic of a feature or features of an embodiment of the disclosure, are understood to mean that the condition or characteristic is defined to within tolerances that are acceptable for operation of an embodiment as described. In addition, the word “or” is considered to be the inclusive “or” rather than the exclusive or, and indicates at least one of, or any combination of items it conjoins.

Descriptions of embodiments of the invention in the present application are provided by way of example and are not intended to limit the scope of the invention. The described embodiments comprise different features, not all of which are required in all embodiments. Some embodiments utilize only some of the features or possible combinations of the features. Variations of embodiments of the invention that are described, and embodiments comprising different combinations of features noted in the described embodiments, will occur to a person having ordinary skill in the art. The scope of the invention is limited only by the claims.

Unless explicitly stated, the method embodiments described herein are not constrained to a particular order in time or chronological sequence. Additionally, some of the described method elements may be skipped, or they may be repeated, during a sequence of operations of a method.

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

Various embodiments have been presented. Each of these embodiments may of course include features from other embodiments presented, and embodiments not specifically described may include various features described herein.

1. A system for a monitoring disease, the system comprising:
a communication device including a memory and a processor, the processor configured to:
obtain information related to a user's medical condition;
determine, while the user is using the communication device, respiratory characteristics of the user based on a recording of a respiration of the user; and
determine a progress of a disease of the user based on the user's medical condition and based on comparing the characteristics to a reference set of characteristics;
wherein the reference set of characteristics is created, by the processor, by obtaining signals related to the user's breathing while the user uses the communication device.

2. The system of claim 1 wherein the processor is further configured to:
determine characteristics of a normal breathing of the user by periodically obtaining respiratory characteristics of the user; and
create the reference set of characteristics based on the characteristics of a normal breathing of the user.

3-6. (canceled)

7. The system of claim 1 wherein the processor is further configured to:
associate the progress of the disease with a score; and
select to perform, based on the score, at least one action.

8. The system of claim 1 wherein the processor is further configured to send the respiratory characteristics of the user to a server and wherein the server is configured to:
rank the breathing characteristics according to a ranking scale; and
based on the rank, select to send a message to at least one of: a physician, a medical institute, a medical stuff member, a caregiver, a family member of the user, and the user.

9. (canceled)

10. The system of claim 1 wherein the processor is further configured to create the reference set of characteristics by:
instructing the user to breath normally; and
obtaining respiratory characteristics of the user.

11. (canceled)

12. The system of claim 1 wherein the processor is further configured to:
generate a baseline based on the respiratory characteristics and based on at least one of:
symptoms exhibited by the user and vital signs of the user; and
determine a progress of a disease based on comparing symptoms exhibited by the user and vital signs of the user to the baseline.

13. The system of claim 1 wherein determining a progress of the disease includes:
identifying, based on comparing the characteristics to the reference set of characteristics, that a threshold was breached, wherein the threshold is defined based on at least one of: a location of the user, an activity of the user, medical history of the user, and recent hospitalization information of the user.

14-16. (canceled)

17. The system of claim 1 comprising:
determining whether or not the user performed a physical activity prior to the determining of the progress of a disease; and
if the user performed a physical activity prior to the determining of the progress of a disease then instructing the user to rest and breathe normally,
recording of a respiration of the user,
extracting one or more respiration characteristics from the recording, and
determining a progress of the disease of the user based on comparing the one or more respiration characteristics to a reference set of characteristics.

18. (canceled)

19. The system of claim 13 wherein the processor is further configured to:
determining nonadherence with a prescribed treatment based on at least one of: comparing respiratory characteristics of the user to a reference set of characteristics, and a report from an adherence system; and
modifying the threshold according to a rule related to the user's medical condition and to the prescribed treatment.

20-21. (canceled)

22. A method of monitoring a disease, the method comprising:
obtaining, by a processor included in a communication device, information related to a user's medical condition;
determining, by the processor and while the user is using the communication device, respiratory characteristics of the user based on a recording of a respiration of the user; and
determining, by the processor, a progress of a disease of the user based on the user's medical condition and based on comparing the characteristics to a reference set of characteristics;
wherein the reference set of characteristics is created, by the processor, by obtaining signals related to the user's breathing while the user uses the communication device.

23. The method of claim 22 further comprising:
determining, by the processor, characteristics of a normal breathing of the user by periodically obtaining respiratory characteristics of the user; and
creating the reference set of characteristics based on the characteristics of a normal breathing of the user.

24-27. (canceled)

28. The method of claim 22 further comprising:
associating, by the processor, the progress of the disease with a score; and
selecting to perform, based on the score, at least one action.

29. The method of claim 22 further comprising:
sending, by the processor, the respiratory characteristics of the user to a server;
ranking, by the server, the breathing characteristics according to a ranking scale; and
based on the rank, selecting to send a message to at least one of: a physician, a medical institute, a medical stuff member, a caregiver, a family member of the user, and the user.

30. (canceled)

31. The method of claim 22 further comprising creating by the processor, the reference set of characteristics by:
instructing the user to breath normally; and
obtaining respiratory characteristics of the user.

32. (canceled)

33. The method of claim 22 further comprising:
generating, by the processor, a baseline based on the respiratory characteristics and based on at least one of: symptoms exhibited by the user and vital signs of the user; and
determining, by the processor, a progress of a disease based on comparing symptoms exhibited by the user and vital signs of the user to the baseline.

34. The method of claim 22 wherein determining a progress of the disease comprises:
identifying, based on comparing the characteristics to the reference set of characteristics, that a threshold was breached, wherein the threshold is defined based on at least one of: a location of the user, an activity of the user, medical history of the user, and recent hospitalization information of the user.

35-37. (canceled)

38. The method of claim 22 comprising:
determining whether or not the user performed a physical activity prior to the determining of the progress of a disease; and
if the user performed a physical activity prior to the determining of the progress of a disease then instructing the user to rest and breathe normally,
recording of a respiration of the user,
extracting one or more respiration characteristics from the recording, and
determining a progress of the disease of the user based on comparing the one or more respiration characteristics to a reference set of characteristics.

39. The method of claim 34 further comprising:
if the threshold was breached then:
instructing the user to breathe normally;
recording of a respiration of the user;
extracting one or more respiration characteristics from the recording; and
determining a progress of the disease of the user based on comparing the one or more respiration characteristics to a reference set of characteristics.

40. The method of claim 34 further comprising:
determining nonadherence with a prescribed treatment based on at least one of: comparing respiratory characteristics of the user to a reference set of characteristics, and a report from an adherence system; and
modifying the threshold according to a rule related to the user's medical condition and to the prescribed treatment.

41-42. (canceled)

43. A method of determining a state of a disease, the method comprising:
obtaining, by a processor included in a communication device, information related to a user's medical condition;
determining, by the processor, respiratory characteristics of the user based on a recording of a respiration of the user; and
determining, by the processor, at least one of: a state, a progress, an improvement and a deterioration of the disease based on the user's medical condition and based on comparing the characteristics to a reference set of characteristics;
wherein the reference set of characteristics is created, by the processor, by obtaining signals related to the user's breathing while the user uses the communication device.
